(19) United States
(12) Patent Application Publication
(10) Pub. No.: US 2025/0127982 A1
(43) Pub. Date: Apr. 24, 2025

(54) FILTER FOR IV SET

(71) Applicant: CareFusion 303, Inc., San Diego, CA (US)

(72) Inventor: Akhil DWIVEDI, Greater Noida West (IN)

(21) Appl. No.: 18/912,132

(22) Filed: Oct. 10, 2024

Related U.S. Application Data
(60) Provisional application No. 63/591,346, filed on Oct. 18, 2023.

(51) Int. Cl. A61M 5/14; A61M 5/165

(52) U.S. Cl. CPC A61M 5/1411; A61M 5/165; A61M 2205/7545

(57) ABSTRACT

Filter assemblies are described herein. A filter assembly includes an outer filter with an outer filter media and an inner filter with an inner filter media. The inner filter media defines an inner flow channel. The inner filter is disposed within the outer filter, defining an annulus between the outer filter and the inner filter. The outer filter media is configured to permit a first flow from the annulus toward an outlet portion of the outer filter and capture particulate from the first flow. The inner filter media is configured to permit a second flow from the annulus toward the inner flow channel and capture particulate from the second flow.

### REFERENCE TO RELATED APPLICATIONS

This application claims priority to U.S. Provisional Patent Application No. 63/591,346entitled “BLOOD SET FILTER DESIGN” filed Oct. 18, 2023, the entire disclosure of which being incorporated herein by this reference.

### FIELD OF THE INVENTION

The present disclosure generally relates to filters, and in particular, to filters for intravenous sets.

### BACKGROUND

Medical treatments often include the infusion of a medical fluid (e.g., blood and/or blood components) to patients using an intravenous (IV) catheter that is connected though an arrangement of flexible tubing and fittings, commonly referred to as an “IV set,” to a source of fluid. During operation, medical fluid can be filtered to prevent the transfer of bacteria, microorganisms, and/or other pathogens.

In some applications, such as blood transfusions for trauma patients, it is desired to transfuse to transfuse blood to the patient in a relatively short period of time. Characteristics of IV set components, such as the filter can impact the flow rate and delivery time of blood to the patient.

### SUMMARY

The disclosed subject matter relates to filters for IV sets. In certain embodiments, filter assemblies are disclosed that comprise an outer filter comprising an outer filter media; and an inner filter comprising an inner filter media defining an inner flow channel, wherein the inner filter is disposed within the outer filter, defining an annulus between the outer filter and the inner filter, wherein the outer filter media is configured to permit a first flow from the annulus toward an outlet portion of the outer filter and capture particulate from the first flow, and the inner filter media is configured to permit a second flow from the annulus toward the inner flow channel and capture particulate from the second flow.

In certain embodiments, a method is disclosed that comprises introducing an inlet flow into a chamber volume; permitting the inlet flow from an inlet portion of the chamber volume through an annulus defined between an outer filter and an inner filter, through the outer filter and into an outlet portion of the chamber volume; capturing particulate from the inlet flow in the outer filter; permitting the inlet flow from the inlet portion of the chamber volume through the annulus, through the inner filter, and into the outlet portion of the chamber volume; and capturing particulate from the inlet flow in the inner filter.

In certain embodiments, a drip chamber assembly is disclosed that comprises a drip chamber comprising a chamber body defining a chamber volume; and a filter disposed within the chamber volume, the filter comprising: a cylindrical outer filter comprising an outer filter media; and a cylindrical inner filter comprising an inner filter media defining an inner flow channel, wherein the inner filter is disposed within the outer filter, defining an annulus between the outer filter and the inner filter, wherein the outer filter media is configured to permit a first flow from an inlet portion of the chamber volume through the annulus and toward an outlet portion of the chamber volume and capture particulate from the first flow, and the inner filter media is configured to permit a second flow from the inlet portion of the chamber volume through the annulus and toward the outlet portion of the chamber volume and capture particulate from the second flow.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 depicts a patient receiving an infusion of a medical fluid using an IV pump.

FIG. 2 illustrates a cross-sectional view of a drip chamber assembly according to certain aspects of the present disclosure.

FIG. 3 illustrates fluid flow through the drip chamber assembly of FIG. 2.

FIG. 4 illustrates a perspective view of a filter of the drip chamber assembly of FIG. 2.

FIG. 5 illustrates a perspective view of a filter of the drip chamber assembly of FIG. 2.

FIG. 6 illustrates a top view of a filter of the drip chamber assembly of FIG. 2.

FIG. 7 illustrates a cross-sectional view of a filter of the drip chamber assembly of FIG. 2.

### DETAILED DESCRIPTION

The disclosed filter provides an annular configuration. The annular filter configuration provides increased filtration area, allowing for increased flow rate through the filter and stable flow through the filter.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to filters for the administration of medical fluid using the disclosed filter, it is to be understood that this description is only an example of usage and does not limit the scope of the claims. Various aspects of the disclosed filter may be used in any application where it is desirable to provide increased flow rate through a filter.

The disclosed filter overcomes several challenges discovered with respect to certain conventional filter devices. One challenge with certain conventional filter devices is that conventional filter media may not provide adequate or desired fluid flow (e.g., blood flow) to a patient. Further, another challenge with certain conventional components is that certain conventional devices may be complex and/or may include numerous component parts. Therefore, certain conventional devices may be difficult to manufacture in a scalable and cost-effective manner. Because certain conventional filter devices may not provide adequate or desired fluid flow and may be difficult to manufacture, the use of certain conventional filtration devices is undesirable.

Therefore, in accordance with the present disclosure, it is advantageous to provide a filter that can allow for increased flow rate without significantly increasing the complexity of the component design. Further, it is advantageous to provide a filter design that allows for efficient utilization of the filter element. Additionally, it is advantageous to provide a filter that provides increased filter area and improved pressure gradient. Also, it is advantageous to provide a filter that reduces fluid (e.g., blood) splash and facilitates stabilizing fluid flow through the filter. Further, it is advantageous to provide a filter design that is easy and cost effective to manufacture and assemble.

Examples of filter and drip chamber assembly that allow for increased fluid flow rate and minimize complexity are now described.

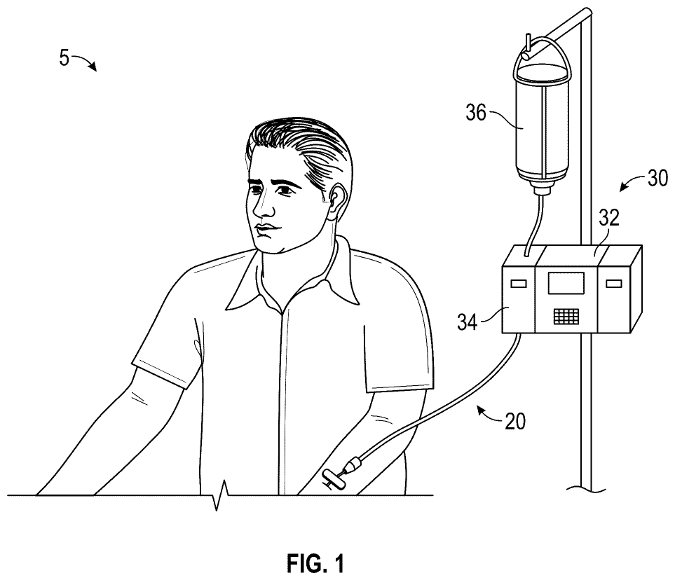

FIG. 1 illustrates a patient 5 receiving an infusion of a medical fluid (e.g., blood) through an IV pump 30 according to certain aspects of the present disclosure. The IV pump 30 comprises a controller 32 and two pump modules 34. An IV set 20 is connected between a container 36 of the medical fluid and the patient 5. Prior to operation, components of the IV set 20 can be primed with medical fluid. Further, during operation, medical fluid delivered to the patient 5 can be filtered to prevent the transfer of bacteria, microorganisms, and/or other pathogens. A filter device as described herein can allow for filtration of the medical fluid delivered to the patient 5. In some embodiments, a filter and/or drip chamber assembly can be disposed in between or in line with tubing of the IV set 20.

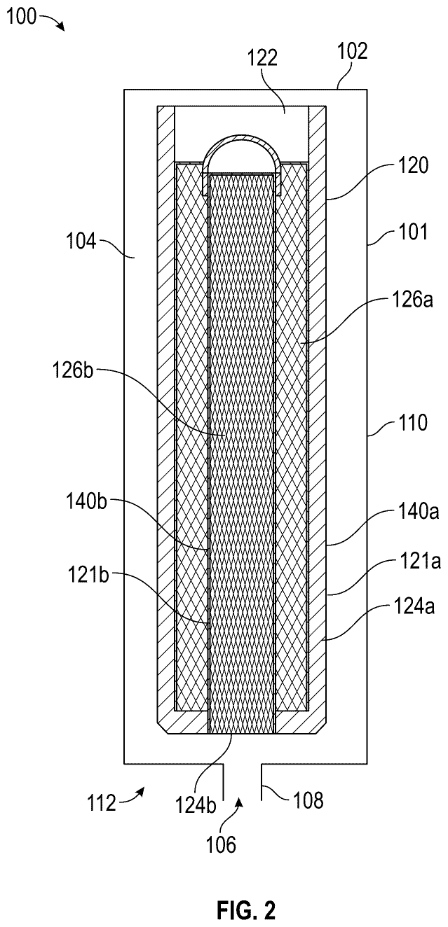

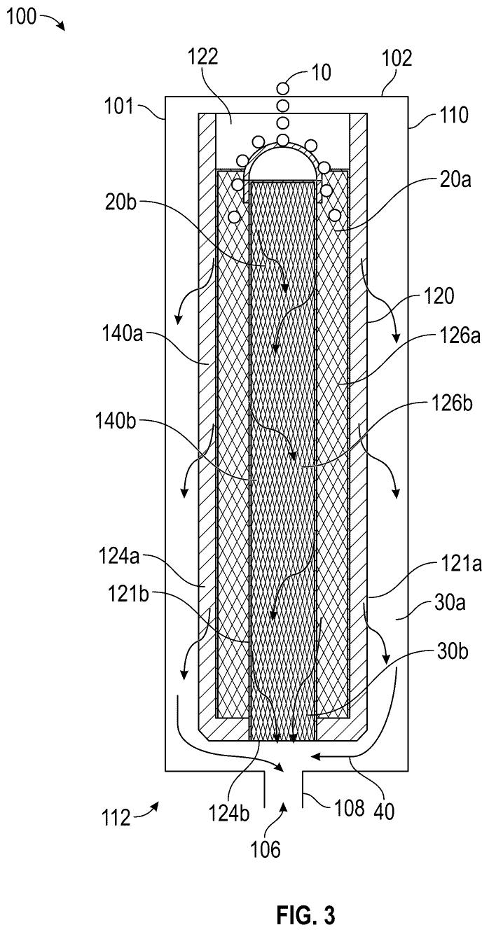

FIG. 2 illustrates a cross-sectional view of a drip chamber assembly 100 according to certain aspects of the present disclosure. FIG. 3 illustrates fluid flow through the drip chamber assembly 100 of FIG. 2. With reference to FIGS. 2 and 3, in the depicted example, the drip chamber assembly 100 allows for the functionality of a drip chamber 101 while additionally allowing for filtration of medical fluid passing therethrough. As described herein, the drip chamber assembly 100 can include a filter 120 disposed within the drip chamber 101.

As illustrated, the drip chamber 101 provides a visual indicator of the flow rate of a medical fluid therethrough. Advantageously, clinicians can monitor and adjust the flow rate of the medical fluid based on the visual indicator provided by the drip chamber 101.

During operation, medical fluid can drip or otherwise flow through the chamber volume 104 defined by the chamber body 110. Medical fluid flow 10 can enter the chamber body 110 through an upper portion or inlet portion 102 defined in the chamber body 110. Fluid flow 40 can exit the chamber body 110 through a lower portion or outlet portion 112. In some embodiments, the outlet portion 112 can include an outlet 108. The outlet lumen 106 formed in the outlet 108 can be in fluid communication with the chamber volume 104. The outlet 108 can be coupled to tubing of the IV set 20.

As fluid passes through the chamber body 110, a clinician can utilize the drip chamber 101 as a visual indicator to observe the dripping or flow of medical fluid therethrough. As can be appreciated the chamber body 110 can be transparent or semi-transparent.

In some embodiments, the chamber body 110 can equalize pressure differentials between the chamber volume 104 and the environment during operation. In some embodiments, the chamber body 110 can be formed from a resilient material to allow the chamber body 110 to be squeezed or compressed to draw in medical fluid for priming of an IV system.

In the depicted example, the drip chamber 101 can draw in medical fluid for priming of an IV system. As can be appreciated, the chamber volume 104 can be filled with a desired volume of medical fluid during the priming operation.

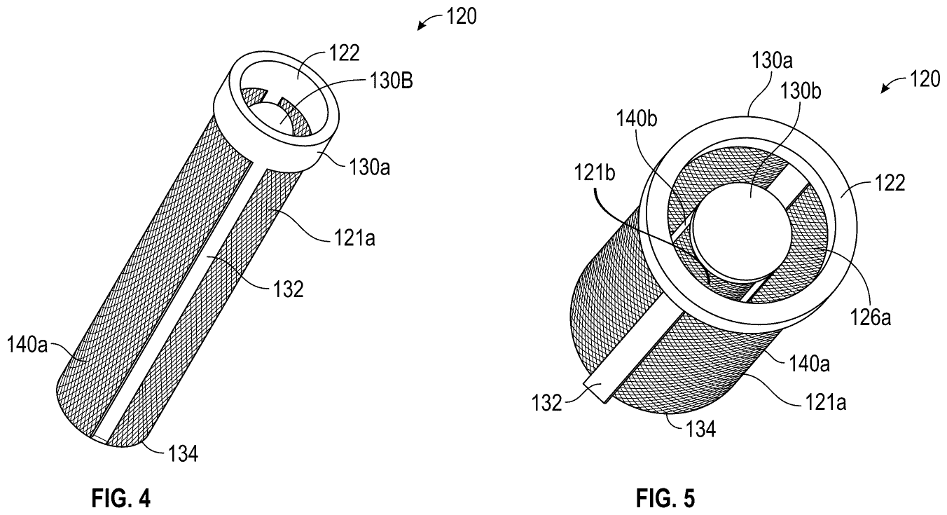

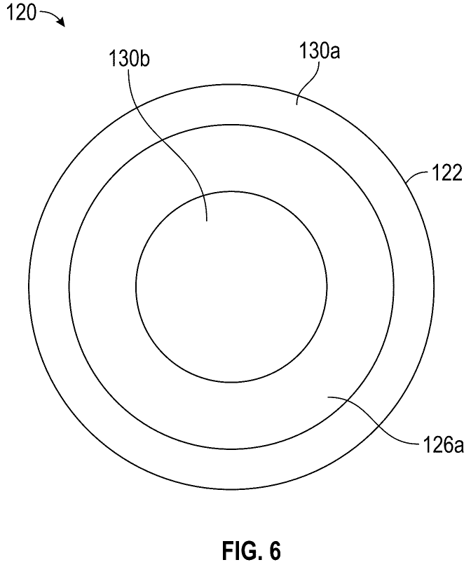

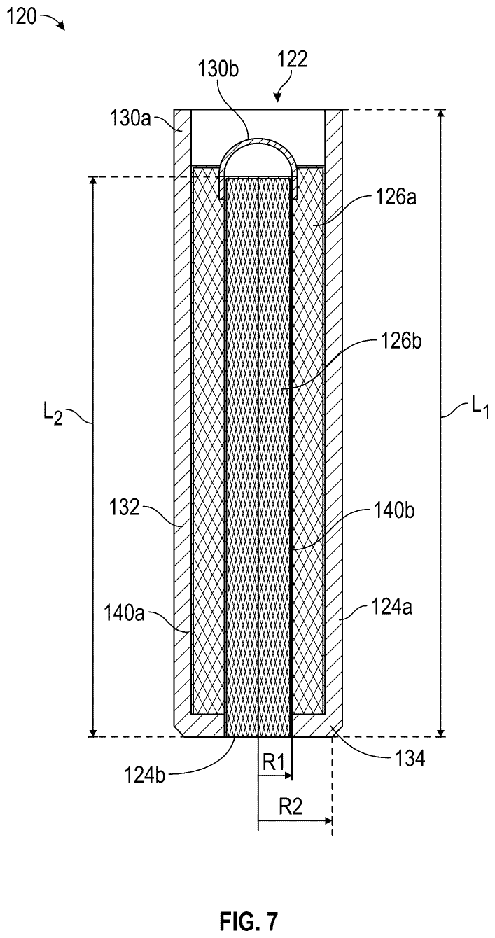

FIG. 4 illustrates a perspective view of a filter 120 of the drip chamber assembly 100 of FIG. 2. FIG. 5 illustrates a perspective view of a filter 120 of the drip chamber assembly 100 of FIG. 2. FIG. 6 illustrates a top view of a filter 120 of the drip chamber assembly 100 of FIG. 2. FIG. 7 illustrates a cross-sectional view of a filter 120 of the drip chamber assembly 100 of FIG. 2. With reference to FIGS. 2-7, during operation, as medical fluid flows through the drip chamber assembly 100, the fluid can be filtered prior to flowing out of the drip chamber 101 and into tubing of the IV set 20. In the depicted example, a filter 120 is disposed within the chamber volume 104 to filter fluid passing through the chamber volume 104.

As illustrated, the filter 120 has an annular configuration. In some embodiments, an inner filter 121b is disposed within an outer filter 121a, defining an annulus 126a therebetween. Optionally, the inner filter 121b can be bonded within or to the outer filter 121a.

With reference to FIG. 3, fluid within the chamber volume 104 can pass through a filter 120 to prevent the transfer of bacteria, microorganisms, and/or other pathogens to the patient. During operation, fluid can flow within an inlet portion of the chamber volume 104 into an inlet portion 122 of the filter 120 as shown by fluid flow 10. From the inlet portion 122 fluid can flow into the annulus 126a between the inner filter 121b and the outer filter 121b. As illustrated by fluid flow 20a, fluid can flow from the annulus 126a outward or toward the outer filter 121a. As shown by fluid flow 30a, fluid can flow through the filter media 140a of the outer filter 121a though an outlet portion 124a of the outer filter 121a. Further, as illustrated by fluid flow 20b, fluid can flow from the annulus 126a inward or toward the inner filter 121b. As shown by the fluid flow 30b, fluid can flow through the filter media 140b of the inner filter 121b and into an inner flow channel 126b defined within the inner filter 121b. Flow can exit the inner flow channel 126b of the inner filter 121b via an outlet portion 124b. As illustrated by fluid flow 40, filtered fluid flow from the outlet portion 124a of the outer filter 121a and the outlet portion 124b of the inner filter 121b can converge in the outlet portion of the chamber volume 104.

In some embodiments, the filter 120 can be seated or spaced apart within the chamber volume 104 to define a flow path between the chamber body 110 and the lower frame 134 of the filter 120. Optionally, the lower frame 134 of the filter 120 can be disposed on raised protrusions defined within the chamber body 110 can promote or otherwise define the flow path between the chamber body 110 and the lower frame 134.

As can be appreciated, a positive pressure differential can direct fluid flow 10 from the inlet portion 122 of the filter 120 through the outer filter 121a and the inner filter 121b and into the outlet portions 124a, 124b of the filter 120. Filtered fluid can flow from the outlet portion 112 through the outlet 108 of the drip chamber 101.

In some embodiments, the annular configuration of the filter 120 can promote the pressure gradient across the annulus 126a, increasing the differential pressure across the outer filter 121a and the inner filter 121b relative to certain conventional cylindrical filter configurations. For example, for a given set of dimensions and fluid flow properties, the velocity distribution of fluid through a cylindrical filter is defined by the following equation:

$$w = \frac{1}{4 μ} ( - \frac{d}{d 𝓏} ( p + γ h ) ( {a}^{2} - {r}^{2} )$$

In contrast, for a given set of dimensions and fluid flow properties, the velocity distribution of fluid through an annular filter is defined by the following equation:

$$w = \frac{1}{4 μ} ( − \frac{d}{d 𝓏} ( p + γ h ) ) [ ( {a}^{2} - {r}^{2} ) + \frac{( {a}^{2} − {b}^{2} ) ln ( \frac{a}{r} )}{ln ( \frac{b}{a} )} ) ]$$

Advantageously, the annular configuration provides an incremental increase in velocity compared to a cylindrical filter configuration for a given set of dimensions and fluid flow properties. As demonstrated in the following equation, energy is conserved in an annular filter design:

$${P}_{h e a d} + {Velocity}_{head} + {Elevation}_{h e a d} = Constant$$

$$( {P}_{1} - {P}_{2} ) + ( {Z}_{1} ‐ {Z}_{2} ) = ( {W}_{2} - {W}_{1} )$$

Therefore, since an annular filter configuration provides an increase in velocity compared to a cylindrical filter configuration, the annular filter configuration similarly provides an increase in pressure differential compared to a cylindrical filter configuration. Therefore, the increased pressure differential or gradient provides an improved flow rate relative to a cylindrical filter configuration.

As described herein, the filter media 140a, 140b can selectively filter the flow through the outer filter 121a and inner filter 121b, respectively. The filter media 140a, 140b can have an average filter opening ranging between 15 to 200 microns. In some embodiments, the average filter opening can range between 180 to 200 microns. Optionally, the filter media 140a, 140b can have pores that vary in size. In some embodiments, the filter media 140a, 140b can be formed from a mesh or non-woven filter material. The filter media 140a, 140b can be formed from a resilient or expandable material. Optionally, the filter media 140a, 140b can be treated with an anti-coagulant.

In some embodiments, the outer filter 121a and the inner filter 121b can have generally cylindrical shapes. As illustrated, the outer filter 121a can have a length L1 that extends along a portion of the length of the drip chamber 101. In some embodiments, the outer filter 121a can have a radius R1 that allows the filter 120 to fit within the chamber volume 104 of the drip chamber 101. Further, the inner filter 121b can have a length L2 that allows the inner filter 121b to fit within the outer filter 121b. Similarly, the inner filter 121b can have a radius R2 that allows the inner filter 121b to fit within the opening of the outer filter 121a and define an annulus 126a therebetween.

In some embodiments, the annular configuration of the filter 120 can provide additional filtration area relative to certain conventional cylindrical filter configurations. For example, for a given set of dimensions, the surface area of filtration for a cylindrical filter is defined by the following equation:

A=2πrl

In contrast, for a given set of dimensions, the surface area of filtration for an annular filter is defined by the following equation:

$$A = 2 π R 1 L 1 + 2 π R 2 L 2$$

Advantageously, the annular configuration provides an incremental increase surface area of filtration (2πR2L2) compared to a cylindrical filter configuration for a given set of dimensions, increasing the flow rate capacity of the annular filter 120.

During operation, fluid flow 20a can enter the outer filter 121a through an inner portion of the filter media 140a via the inlet portion of the chamber volume 104. The flow 30a can move radially outward through the filter media 140a and downward into the outlet portion of the chamber volume 110. Similarly, fluid flow 20b can enter the inner filter 121b through an outer portion of the filter media 140b via the annulus 126a. The flow 30b can move radially inward through the filter media 140b and into the inner flow channel 126b. The flow from the inner flow channel 126b can flow downward into the outlet portion of the chamber volume 110.

In some embodiments, the filter 120 is supported by a filter frame. As illustrated, the outer filter media 140a can be supported by an upper frame 130a and/or a lower frame 134. The upper frame 130a and/or the lower frame 134 can maintain the general shape of the filter media 140a. In some embodiments, ribs or columns 132 can connect the upper frame 130a and the lower frame 134 to provide additional support or rigidity to the frame and filter 120. Optionally, the upper frame 130a and/or the lower frame 134 can seal against the chamber body 110 to prevent flow from bypassing the filter media 140a. The upper frame 130a and/or the lower frame 134 can have a generally resilient construction to maintain sealing contact with the chamber body 110 during deformation of the drip chamber 101. Further, the lower frame 134 can act as an endplate to prevent flow from bypassing the filter media 140a. In some applications, the filter media 140a and/or the filter media 140b can be bonded to the lower frame 134.

In some embodiments, the inner filter 121b includes a rounded top 130b to direct fluid flow. During operation, the rounded top 130b can direct fluid flow into the annulus 126a, stabilizing fluid flow and preventing splashing. In some embodiments, the rounded top 130b is bonded to the filter media 140b of the inner filter 121b.

Optionally, the filter 120 is captured by features and/or geometry of the drip chamber 101. In some embodiments, the filter 120 is coupled to the drip chamber 101 via fasteners and/or an adhesive. The filter 120 may also be free floating relative to the drip chamber 101.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A filter assembly comprising:
an outer filter comprising an outer filter media; and
an inner filter comprising an inner filter media defining an inner flow channel, wherein the inner filter is disposed within the outer filter, defining an annulus between the outer filter and the inner filter,
wherein the outer filter media is configured to permit a first flow from the annulus toward an outlet portion of the outer filter and capture particulate from the first flow, and the inner filter media is configured to permit a second flow from the annulus toward the inner flow channel and capture particulate from the second flow.

2. The filter assembly of claim 1, wherein the outer filter and the inner filter are cylindrical.

3. The filter assembly of claim 1, wherein the outer filter media is configured to permit the first flow from the annulus radially outward toward through the outer filter media.

4. The filter assembly of claim 1, wherein the inner filter media is configured to permit the second flow from the annulus radially inward through the inner filter media.

5. The filter assembly of claim 1, wherein the inner filter has an inner filter length less than an outer filter length of the outer filter.

6. The filter assembly of claim 1, wherein the inner filter has an inner filter radius less than an outer filter radius of the outer filter.

7. The filter assembly of claim 1, further comprising a frame to support the outer filter media, wherein the frame directs the first flow and the second flow toward the annulus.

8. The filter assembly of claim 7, wherein an upper portion of the frame directs the first flow and the second flow toward the annulus.

9. The filter assembly of claim 8, further comprising a lower portion of the frame, wherein the lower portion of the frame prevents the first flow and the second flow from bypassing the outer filter media and the inner filter media.

10. The filter assembly of claim 9, wherein the upper portion of the frame and the lower portion of the frame are coupled by a column.

11. The filter assembly of claim 9, wherein the inner filter media is bonded to the lower portion of the frame.

12. The filter assembly of claim 1, further comprising a rounded top coupled to an upper portion of the inner filter media, wherein the rounded top is configured to direct the first flow and the second flow into the annulus.

13. The filter assembly of claim 12, wherein the rounded top is bonded to the inner filter media.

14. A method comprising:
introducing an inlet flow into a chamber volume;
permitting the inlet flow from an inlet portion of the chamber volume through an annulus defined between an outer filter and an inner filter, through the outer filter and into an outlet portion of the chamber volume;
capturing particulate from the inlet flow in the outer filter;
permitting the inlet flow from the inlet portion of the chamber volume through the annulus, through the inner filter, and into the outlet portion of the chamber volume; and
capturing particulate from the inlet flow in the inner filter.

15. The method of claim 14, further comprising permitting the inlet flow from the annulus radially outward through the outer filter.

16. The method of claim 14, further comprising permitting the inlet flow from the annulus radially inward through the inner filter.

17. The method of claim 14, further comprising permitting the inlet flow from the annulus through the inner filter and into an inner flow channel defined by the inner filter.

18. A drip chamber assembly, comprising:
a drip chamber comprising a chamber body defining a chamber volume; and
a filter disposed within the chamber volume, the filter comprising:
a cylindrical outer filter comprising an outer filter media; and
a cylindrical inner filter comprising an inner filter media defining an inner flow channel, wherein the inner filter is disposed within the outer filter, defining an annulus between the outer filter and the inner filter,
wherein the outer filter media is configured to permit a first flow from an inlet portion of the chamber volume through the annulus and toward an outlet portion of the chamber volume and capture particulate from the first flow, and the inner filter media is configured to permit a second flow from the inlet portion of the chamber volume through the annulus and toward the outlet portion of the chamber volume and capture particulate from the second flow.

19. The drip chamber assembly of claim 18, wherein the outer filter media is configured to permit the first flow from the annulus radially outward toward through the outer filter media.

20. The drip chamber assembly of claim 18, wherein the inner filter media is configured to permit the second flow from the annulus radially inward through the inner filter media.
